(19) United States
(12) Patent Application Publication
(10) Pub. No.: US 2025/0381020 A1
(43) Pub. Date: Dec. 18, 2025

(54) IMPLANTABLE PROSTHESIS

(71) Applicant: Davol Inc., Warwick, RI (US)

(72) Inventors: Ian K. Parker, Bristol, RI (US); Melissa Bowley, Newport, RI (US); Greg Daniel Learn, West Warwick, RI (US)

(73) Assignee: Davol Inc., Warwick, RI (US)

(21) Appl. No.: 18/877,224

(22) PCT Filed: Jun. 29, 2023
(86) PCT No.: PCT/US2023/026549

§ 371 Date: Dec. 19, 2024

Related U.S. Application Data
(60) Provisional application No. 63/357,152, filed on Jun. 30, 2022.
(60) Provisional application No. 63/357,175, filed on Jun. 30, 2022.

(51) Int. Cl. A61F 2/00

(52) U.S. Cl. CPC A61F 2/0063

(57) ABSTRACT

An implantable prosthesis for preventing, reinforcing and/or mending an anatomical defect, including a parastomal hernia which may occur following the formation of a stoma or ostomy opening, such as formed in conjunction with an ileostomy or colostomy. The prosthesis may include a body of biocompatible material having a preformed three-dimensional channel configured to receive a portion of a lateralized bowel formed in conjunction with a parastomal hernia repair or ostomy creation. An adhesion resistant barrier may be located on an inner side of the channel to isolate the portion of the lateralized bowel from a tissue infiltratable surface of the body to reduce or eliminate potential adhesions between the body and the lateralized bowel. A portion of the channel may include a flared segment extending inwardly from the outer periphery to reduce pressure on the portion of the lateralized bowel at the open end of the channel.

### RELATED APPLICATIONS

This application claims the benefits of U.S. application No. 63/357,152, filed Jun. 30, 2022 and U.S. application No. 63/357,175, filed Jun. 30, 2022, both of which are hereby incorporated by reference in its entirety.

### FIELD

The present invention relates to an implantable prosthesis, and more particularly to a prosthesis for mending defects and weaknesses of soft tissue and muscle walls.

### BACKGROUND

A defect in a muscle or tissue wall, such as a hernia, is commonly repaired with an implantable prosthesis that is configured to cover and/or fill the defect.

For some procedures, an implantable repair fabric, such as a mesh fabric, is sutured, stapled, tacked, or otherwise provisionally anchored in place over, under or within the defect. Tissue integration with the fabric, such as tissue ingrowth into and/or along the mesh fabric, eventually completes the repair.

Various repair fabrics are known and used for repairing soft tissue and muscle wall defects. Examples of implantable fabrics that have been successfully used in soft tissue and muscle wall repair include BARD Soft Mesh, BARD Mesh, PHASIX Mesh and PHASIX ST Mesh, available from Davol, a division of Becton Dickinson. Such fabrics are fabricated from monofilaments that are knitted into a mesh having pores or interstices that promote tissue ingrowth and integration with the fabric. Some fabrics have adhesion resistance properties.

A parastomal hernia is a type of incisional hernia that occurs at or adjacent to a stoma. It is a complication associated with the creation of an ileostomy, colostomy, or ileal conduit stoma. Parastomal hernias may be repaired using various techniques, including the Sugarbaker technique. This repair involves a surgeon performing an open surgery, such as a laparotomy, or a minimally invasive procedure, such as laparoscopic or robotic surgery. Following hernia reduction, sac resection and reduction of the stoma trephine, the ostomy opening is covered with an intraperitoneally placed prosthetic mesh that is sutured to the fascia. The bowel is lateralized and secured between the mesh and the peritoneum, thereby lateralizing the forces which press the bowel ventrally onto the abdominal wall, shifting them from pushing up toward the defect and causing these forces to press ventrally against an intact abdominal wall.

It is an object of the invention to provide a prosthesis and method for preventing and/or mending defects in soft tissue and muscle walls, including a parastomal hernia.

### SUMMARY

The present disclosure relates to an implantable prosthesis and method for reinforcing, preventing and/or mending an anatomical defect, such as a tissue or muscle wall defect, including a parastomal hernia.

According to one aspect, an implantable prosthesis comprises a body of implantable. biologically compatible material, a channel preformed on the body and an adhesion resistant barrier. The body has a preformed three-dimensional configuration including a first surface and a second surface opposite the first surface. The first surface is tissue infiltratable and the second surface is adhesion resistant. The channel has a three-dimension configuration to receive a portion of a lateralized bowel and includes an inner side formed by a portion of the first surface and an outer side formed by a portion of the second surface. The adhesion resistant barrier is located on the inner side of the channel to isolate the portion of the lateralized bowel from the first surface.

According to another aspect, an implantable prosthesis comprises a body of implantable, biologically compatible material, a channel on the body and an adhesion resistant barrier. The body has a circular configuration and includes a first surface and a second surface opposite the first surface. The first surface is tissue infiltratable and the second surface is adhesion resistant. The channel is configured to receive a portion of a lateralized bowel and includes an inner side formed by a portion of the first surface and an outer side formed by a portion of the second surface. The adhesion resistant barrier is located on the inner side of the channel to isolate the portion of the lateralized bowel from the first surface.

According to another aspect, a method is provided for reinforcing a stoma, repairing a parastomal hernia or preventing a parastomal hernia. The method comprises an act of: (a) placing a prosthesis intraperitoneally over an ostomy. The prosthesis includes a body of implantable, biologically compatible material and a channel preformed on the body. The channel has a three-dimension configuration to receive a portion of a lateralized bowel. The body includes a first surface and a second surface opposite the first surface. The first surface is tissue infiltratable and the second surface is adhesion resistant. The channel includes an inner side formed by a portion of the first surface and an outer side formed by a portion of the second surface. An adhesion resistant barrier is located on the inner side of the channel. The method further comprises acts of (b) placing the preformed channel over a portion of a lateralized bowel extending from the ostomy with the adhesion resistant barrier isolating the portion of the lateralized bowel from the inner side of the channel, and securing the prosthesis to fascia.

According to another aspect, a method is provided for reinforcing a stoma, repairing a parastomal hernia or preventing a parastomal hernia. The method comprises an act of: (a) placing a prosthesis intraperitoneally over an ostomy. The prosthesis includes a body of implantable, biologically compatible material and a channel preformed on the body. The channel is configured to receive a portion of a lateralized bowel. The body has a circular configuration and includes a first surface and a second surface opposite the first surface. The first surface is tissue infiltratable and the second surface is adhesion resistant. The channel includes an inner side formed by a portion of the first surface and an outer side formed by a portion of the second surface. An adhesion resistant barrier is located on the inner side of the channel. The method further comprises acts of (b) placing the channel over a portion of a lateralized bowel extending from the ostomy with the adhesion resistant barrier isolating the portion of the lateralized bowel from the inner side of the channel, and securing the prosthesis to fascia.

According to another aspect, an implantable prosthesis comprises a body of implantable, biologically compatible material and a channel preformed on the body. The body has a preformed three-dimensional configuration and includes a first surface and a second surface opposite the first surface. The body also includes an outer periphery. The channel is preformed on the body and has a three-dimension configuration to receive a portion of a lateralized bowel. The channel includes an inner side formed by a portion of the first surface and an outer side formed by a portion of the second surface. The channel extends from the outer periphery to an inner region of the body and includes an open end at the outer periphery and a closed end at the inner region. A portion of the channel includes a flared segment extending from the outer periphery toward the closed end.

According to another aspect, an implantable prosthesis comprises a body of implantable, biologically compatible material and includes a first surface and a second surface opposite the first surface. The body includes a lateral portion and a medial portion extending from the lateral portion and further includes an outer periphery. The implantable prosthesis further comprises a channel preformed on the lateral portion of the body. The channel has a three-dimensional configuration to receive a portion of a lateralized bowel. The channel includes an inner side formed by a portion of the first surface and an outer side formed by a portion of the second surface. The channel extends from the outer periphery to an inner region of the body, and includes an open end at the outer periphery and a closed end at the inner region. The lateral portion is configured to be placed at a stoma site to prevent and/or repair a parastomal hernia, and the medial portion is configured to be placed at a midline abdominal incision when the lateral portion is placed at the stoma site.

The foregoing is a non-limiting summary of the disclosure, which is defined by the attached claims. Other aspects, embodiments and/or features will become apparent from the following description.

Various embodiments of the present disclosure may provide certain advantages and may overcome certain drawbacks of prior prostheses. Embodiments of the disclosure may not share the same advantages, and those that do may not share them under all circumstances.

### BRIEF DESCRIPTION OF DRAWINGS

Aspects of the disclosure are described below, by way of example, with reference to the accompanying drawings in which like numerals reference like elements, and wherein:

FIG. 1 is a top, perspective view of an implantable prosthesis according to one embodiment;

FIG. 2 is a bottom, perspective view of the implantable prosthesis of FIG. 1;

FIG. 3 is a top, planar view of the implantable prosthesis of FIGS. 1-2;

FIG. 4 is a side, elevational view of the implantable prosthesis of FIGS. 1-3 illustrating the open end of the channel;

FIG. 5 is a cross sectional view taken along section line 5-5 of FIG. 3 illustrating the length of the channel of the prosthesis;

FIG. 6 is a schematic illustration of a parastomal hernia repair with the implantable prosthesis of FIGS. 1-5 positioned over the ostomy opening and lateralized bowel;

FIG. 7 is a schematic, cross sectional illustration of a parastomal hernia repair employing the implantable prosthesis of FIGS. 1-5;

FIG. 8 is a top, perspective view of an implantable prosthesis according to one embodiment;

FIG. 9 is a top, planar view of the implantable prosthesis of FIG. 8;

FIG. 10 is a side, elevational view of the implantable prosthesis of FIGS. 8-9;

FIG. 11 is an exploded view of the implantable prosthesis of FIG. 8 according to one embodiment;

FIG. 12 is an exploded view of an implantable prosthesis of FIG. 8 according to another embodiment;

FIG. 13 is a top, perspective view of an implantable prosthesis according to one embodiment;

FIG. 14 is a top, planar view of the implantable prosthesis of FIG. 13;

FIG. 15 is a side, elevational view of the implantable prosthesis of FIGS. 13-14; and

FIG. 16 is an exploded view of the implantable prosthesis of FIG. 13 according to one embodiment.

### DETAILED DESCRIPTION

It should be understood that aspects of the disclosure are described herein with reference to the figures, which show illustrative embodiments in accordance with aspects of the disclosure. The illustrative embodiments described herein are not necessarily intended to show all aspects of the disclosure, but are used to describe one or more illustrative embodiments. Thus, aspects of the disclosure are not intended to be construed narrowly in view of the illustrative embodiments. It should be appreciated, then, that the various concepts and embodiments discussed herein may be implemented in any of numerous ways, as the disclosed concepts and embodiments are not limited to any particular manner of implementation. In addition, it should be understood that aspects of the disclosure may be used alone or in any suitable combination with other aspects of the disclosure.

The disclosure is directed to an implantable prosthesis for preventing, reinforcing and/or mending an anatomical defect, and is particularly suitable for preventing, reinforcing and/or mending defects in, and weaknesses of, soft tissue and muscle walls or other anatomical regions. The phrase “mending a defect” includes acts of repairing, augmenting, and/or reconstructing a defect and/or a potential defect. For ease of understanding, and without limiting the scope of the invention, the prosthesis is described below particularly employed in connection with a parastomal hernia which may potentially occur following the formation of a stoma or ostomy opening, such as formed in conjunction with an ileostomy, colostomy, or ilcal conduit stoma. It should be understood, however, that the prosthesis is not so limited and may be employed in other anatomical procedures, as should be apparent to one of skill in the art. For example, and without limitation, the prosthesis may be employed with a ventral hernia repair, inguinal hernias, chest or abdominal wall reconstruction, or large defects, such as those that may occur in obese patients. The prosthesis may include one or more features, each independently or in combination, contributing to such attributes.

The disclosure is more particularly directed to a prosthesis which includes a body of biologically compatible material, such as a repair fabric, which is configured to cover or extend across the defect opening or weakness when the body is placed against the defect. The prosthesis may be in the form of a patch, although the prosthesis may employ other configurations as should be apparent to one of skill in the art. The patch may have a planar or non-planar configuration suitable for a particular procedure employed for mending a defect.

The prosthesis may include a body having a preformed three-dimensional configuration with a preformed channel configured to receive a portion of a lateralized bowel formed in conjunction with a parastomal hernia repair. The preformed channel may allow a surgeon to suture or tack the prosthesis along the edge of the channel without compressing the lateralized bowel while achieving a snug fit which eliminates a potential gap which could permit entry of additional bowel and/or without an inadvertent enterotomy.

The body may include a first surface and a second surface opposite the first surface with the first surface being tissue infiltratable and the second surface being adhesion resistant. The channel may include an inner side formed by a portion of the first surface and an outer side formed by a portion of the second surface. An adhesion resistant barrier may be located on the inner side of the channel to isolate the portion of the lateralized bowel from the first surface. In this manner, the barrier layer may reduce, if not eliminate, potential adhesions between the body and the lateralized bowel.

The body may include an outer periphery with the channel extending from the outer periphery to an inner region of the body. The channel may include an open end at the outer periphery and a closed end opposite the open end and at the inner region of the body. The open end of the channel may include a flared segment configured to minimize pressure along the outer periphery which could otherwise potentially lead to erosion of the body into the intestine. The flared segment may be configured to reduce sharp angulation or kinking of the bowel along the outer periphery which could otherwise potentially lead to intestinal obstruction.

In one embodiment, the prosthetic body may be configured with a circular shape with the channel extending in a radial direction from the outer periphery to the center of the body. The channel may have a partial tubular configuration with a semicircular shape within a plane transverse to the length of the channel. The closed end of the channel may have a partial hemispherical shape and be located proximate the center of the body. However, the prosthesis may employ a body having any suitable shape for a particular application. For example, and without limitation, the body may be configured with a quadrilateral shape, such as a square or rectangle, a triangular shape, a polygonal shape, an ovoid shape, or an elliptical shape. The portion of the body excluding the channel may have a planar configuration or a curved configuration, such as a concave or convex shape relative to the channel.

In one embodiment, the prosthetic body may include a medial portion and a lateral portion extending from the medial portion. The lateral portion may be configured to cover the site of a stoma and include the preformed channel for receiving a lateralized bowel. The medial portion may be configured to support a midline laparotomy incision, such as an incision through the linea alba, created during an open surgical procedure, for example, to repair a ventral hernia. The lateral portion may have a circular shape and the medial portion may have a quadrilateral shape, such as a rectangular shape, which may be aligned with the incision.

According to one aspect, the body and/or the channel barrier may be formed of a bioresorbable material. However, it is to be appreciated that the prosthesis may be formed of non-resorbable materials or a combination of resorbable and non-resorbable materials.

According to one aspect, the body and/or the barriers may be formed of materials with translucent or transparent properties so as to facilitate visualization of the intestine and soft tissue behind the prosthesis during fixation. For example, and without limitation, the translucency may be achieved using transparent fibers and/or knits having an adequate pore size to allow visualization through the mesh, such as VISILEX mesh available from Davol, Inc. If desired, different transparent or translucent components may be colored with different tints to provide contrast on a surgical field and facilitate visualization of those components, without sacrificing translucency or visibility of the tissues behind the prosthesis

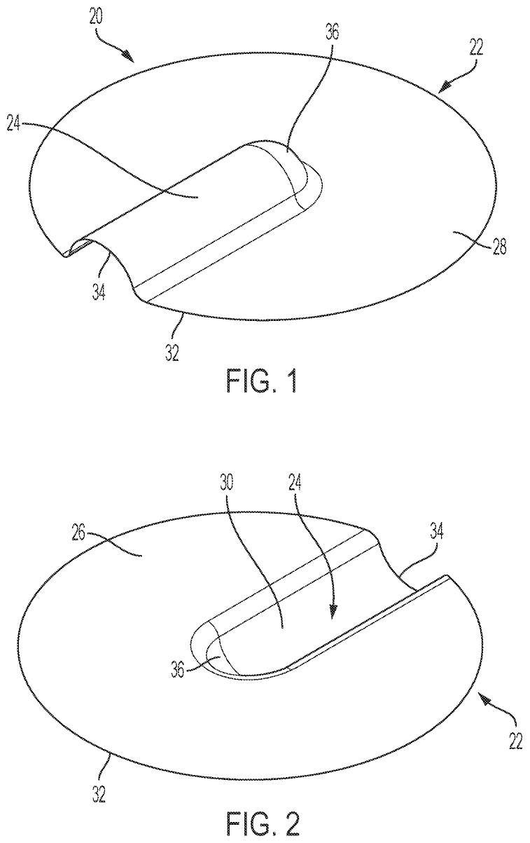

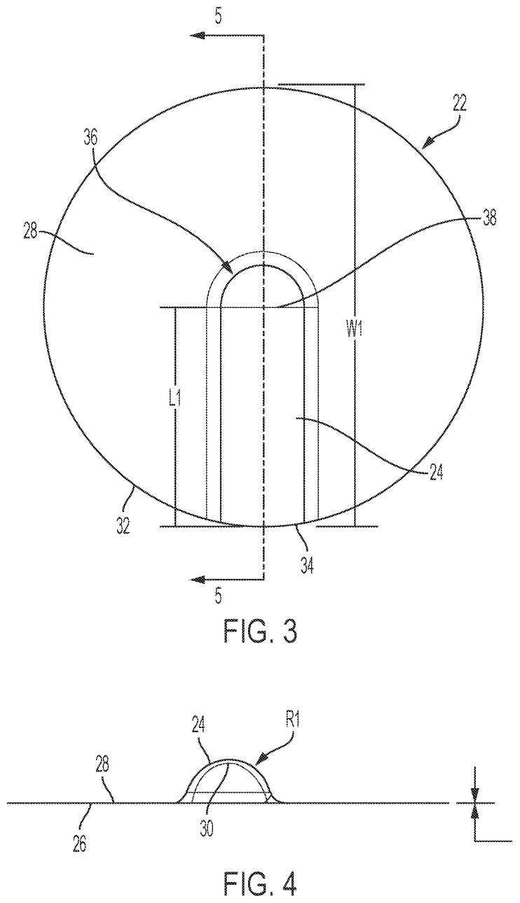

FIGS. 1-3 illustrate one embodiment of a prosthesis for covering, reinforcing and/or mending tissue and muscle wall defects including, without limitation, a defect in the peritoneum which may be formed during a preperitoneal hernia repair procedure. The prosthesis 20 may include a body 22 of implantable, biologically compatible material and a channel 24 preformed with the body. In this manner, the prosthesis may have a preformed three dimensional configuration.

In one embodiment, the body 22 may include a repair fabric which is relatively flexible, thin and light weight and meets the performance and physical characteristics for covering, reinforcing and/or mending soft tissue and muscle wall defects. The body 22 may be configured with a size and/or shape suitable to cover or extend across the opening or weakness in tissue and/or muscle when the body is placed against the tissue and/or muscle wall with the defect.

The body may include a first surface 26 (FIG. 2) and a second surface 28 (FIG. 1) opposite the first surface. In one embodiment, the first surface 26 may be tissue infiltratable and the second surface 28 may be adhesion resistant. In this manner, and without limitation, the prosthesis may be positioned at a repair site with the first surface 26 facing soft tissue and/or muscle for receiving tissue ingrowth and the second surface 28 facing the viscera, or other organs, tissue and/or muscle, to avoid tissue adhesions to the body.

In one embodiment, the channel 24 may be configured to receive a portion of a lateralized bowel formed in conjunction with a parastomal hernia repair, such as formed with the Sugarbaker technique. The channel 24 may include an inner side formed by a portion of the first surface 26 and an outer side formed by a portion of the second surface 28.

For some applications, it may be desirable to reduce the incidence of adhesions between the prosthesis and bowel received in the channel. In one embodiment, the prosthesis may include an adhesion resistant barrier 30 located on the inner side of the channel to isolate the portion of the lateralized bowel from the first surface 26. In this manner, the barrier layer 30 may reduce, if not eliminate, potential adhesions between the body and the lateralized bowel.

In one embodiment illustrated in FIGS. 1-3, the channel 24 may be arranged to extend from the outer periphery 32 to an inner region of the body. The channel may include an open end 34 at the outer periphery and a closed end 36 opposite the open end. The closed end 36 of the channel may be configured and arranged to overlie the ostomy opening through which passes the bowel. As illustrated in FIG. 4, the channel 24 may have a partial tubular configuration with a semicircular shape within a plane oriented perpendicular to the length of the channel. The closed end 36 of the channel may be configured with a partial hemispherical shape and be located proximate the center 38 of the body.

In one embodiment illustrated in FIGS. 1-3, the prosthetic body 22 may be configured with a circular shape. However, it is to be appreciated that the prosthesis may employ a body having any suitable shape for a particular application. For example, and without limitation, the body may be configured with a quadrilateral shape, such as a square or rectangle, a triangular shape, a polygonal shape, an ovoid shape, or an elliptical shape.

As illustrated in FIGS. 1-3, the preformed channel 24 may extend in a radial direction from the outer periphery to the center of the body. The closed end 36 of the channel may be formed about the center of the body. As illustrated, the channel may be configured to extend linearly from the open end 34 to the closed end 36. However, it is to be appreciated that the channel may be preformed to have any suitable configuration as should be apparent to one of skill.

The body and the channel may be configured to have any shape and/or size suitable for a particular application and/or to accommodate a lateralized bowel of various sizes as should be understood by one of skill in the art. In one embodiment, the body may have a maximum width W1 from approximately 5.5 inches (14 cm) to approximately 9.8 inches (25 cm) depending on the size of the bowel being covered by the prosthesis. Similarly, the linear channel may have a length from approximately 2.7 inches (6.9 cm) to approximately 4.9 inches (12.5 cm). The channel may have a radius of curvature R1 from approximately 0.59 inches (1.5 cm) to approximately 1.8 inches (4.5 cm) about its longitudinal axis and a full radius of curvature from approximately 0.59 inches (1.5 cm) to approximately 1.8 inches (4.5 cm) about the center of the body to form the closed end of the channel.

For a body with a circular shape, the maximum width corresponds with the diameter.

In one embodiment for a small bowel, the body may have a diameter of approximately 5.9 inches (15 cm) to approximately 7.9 inches (20 cm) and a linear channel with a length L1 of approximately 3.0 inches (7.6 cm) to approximately 4.0 inches (10 cm) along its longitudinal axis from the outer periphery 32 to the center 38 of the body. The channel may have a radius of curvature R1 of approximately 0.59 inches (1.5 cm) about its longitudinal axis and a full radius of approximately 0.59 inches (1.5 cm) about the center of the body to form the closed end of the channel. In one embodiment, the body may have a diameter of approximately 6.0 inches (15.2 cm) and the channel may have a length L1 of approximately 3.0 inches (7.6 cm).

In one embodiment for a large bowel, the body may have a diameter of approximately 7.1 inches (18 cm) to approximately 8.7 inches (22 cm) and a linear channel with a length L1 of approximately 3.5 inches (9 cm) to approximately 4.0 inches (11 cm) along its longitudinal axis from the outer periphery 32 to the center 38 of the body. The channel may have a radius of curvature R1 of approximately 1.18 inches (3.0 cm) about its longitudinal axis and a full radius of curvature of approximately 1.18 inches (3.0 cm) about the center of the body to form the closed end of the channel.

In one embodiment for a cecum, the body may have a diameter of approximately 8.2 inches (21 cm) to approximately 9.8 inches (25 cm) and a linear channel with a length L1 of approximately 4.1 inches (10.5 cm) to approximately 4.9 inches (12.5 cm) along its longitudinal axis from the outer periphery 32 to the center 38 of the body. The channel may have a radius of curvature R1 of approximately 1.77 inches (4.5 cm) about its longitudinal axis and a full radius of curvature of approximately 1.77 inches (4.5 cm) about the center of the body to form the closed end of the channel.

In one embodiment, the portion of the body 22 excluding the channel 24 may have a planar configuration prior to implantation. The body may have sufficient flexibility to conform to the anatomy adjacent the ostomy upon implantation to repair the hernia defect. In other embodiments, the portion of the body extending outwardly from the channel may have a non-planar configuration, such as a preformed curved configuration, including a concave or convex shape relative to the channel, which may also have a desired amount of flexibility.

The preformed channel 24 may be formed in the body using a vacuum thermoforming technique. In this manner, a sheet of body material may be compressed using a mold or forming tool by applying heat and vacuum so as to thermally and/or plastically deform the sheet and form the channel in a desired shape and size defined by the mold or forming tool. The heat and vacuum may be applied directly to the sheet of material, or indirectly, for example, by placing a sheet of body material between the forming tool and a heated sheet of elastic material, such as rubber. A vacuum removes the air and stretches the elastic material and body material over or into the forming tool. This process may be repeated using progressively larger forms to achieve the final geometry for the channel and other features of the body. If desired, the body may also be preformed in a similar manner to have a non-planar configuration. It is to be appreciated that other fabrication processes, such as cold forming using a rigid mold, may be employed to preform the channel and/or non-planar body as should be apparent to one of skill in the art.

The body 22 may include a mesh fabric and may employ a knit construction that provides openings or pores to allow tissue infiltration to incorporate the prosthesis. If desired, the body 22 may employ a warp-knitted mesh to allow for trimming of the areas adjacent or opposite the channel to optimize support of the lateralized intestine and the ostomy trephine. The repair fabric may also have sufficient flexibility to promote an easy reduction in size for entry into the subject. In this manner, the flexible fabric may be collapsed into a slender configuration, such as a roll, which can be supported in, and advanced through, a narrow vacuumopic cannula for use in laparoscopic or robotic procedures.

In one embodiment, the body 22 of the prosthesis may be formed from a layer of PHASIX ST mesh (available from Davol, Inc.) which is a resorbable repair fabric having a first surface 26 that supports functional healing via tissue ingrowth and a second surface 28 with a hydrogel barrier that is adhesion resistant to minimize tissue attachment. When implanted, the mesh promotes rapid tissue or muscle ingrowth into and around the mesh structure while minimizing potential attachment to adjacent viscera. The body eventually becomes resorbed at which time the prosthesis is no longer necessary to separate the viscera on one side of the prosthesis from the abdominal wall on the opposite side of the prosthesis. For example, and without limitation, the body may not be resorbed until such time that the parastomal hernia repair has sufficiently healed so as to no longer be susceptible to adhesions with the viscera. It is to be appreciated that any suitable repair fabric providing tissue ingrowth and adhesion resistance may be employed for the body as should be understood by one of skill.

In another embodiment, the body 22 of the prosthesis may be formed from a layer of a softer or more elastic P4HB fabric, such as Galaflex Lite. A softer texture may be desired to be gentler on any intestines that the prosthesis may contact. Furthermore, greater elasticity of the material may allow the prosthesis to snugly support the lateralized intestine and the ostomy trephine while avoiding excessive constriction.

In one embodiment, the channel barrier layer 30 may be formed from a layer of SEPRAFILM (available from Baxter Advanced Surgery) which is a resorbable, translucent and hydrophilic adhesion barrier which reduces the incidence of adhesions. It is composed of modified sodium hyaluronate (HA) and carboxymethylcellulose (CMC). The barrier layer eventually becomes resorbed at which time it is no longer necessary to separate the lateralized bowel on one side of the barrier from the repair fabric on the opposite side of the barrier. For example, and without limitation, the barrier layer may not be resorbed until such time that the repair fabric has been resorbed and/or is otherwise no longer susceptible to adhesions with the bowel. It is to be appreciated that any suitable barrier material may be employed for the channel barrier as should be understood by one of skill.

For some applications, the prosthesis 20 may be formed from two or more layers of biocompatible material. For example, and without limitation, the body 22 of the prosthesis may include a first or ingrowth layer of knitted mesh fabric and a second or barrier layer of adhesion resistant material overlying a surface of the mesh fabric. The barrier layer may be separate from and attached to the fabric layer using any suitable fastening technique as should be apparent to one of skill in the art. For example, and without limitation, the mesh fabric and the barrier layer may be joined together by any one or more of suturing, stitching, in situ polymerization, solvent welding, ultrasonic welding, lamination and/or overmolding.

Examples of resorbable surgical materials and/or fabrics which may be utilized for the first or ingrowth layer and are suitable for tissue or muscle reinforcement and defect correction include, but are not limited to, Poly-4-hydroxybutyrate (P4HB), such as PHASIX Mesh (available from Davol, Inc.), polyglactin (VICRYL—available from Ethicon, Inc.) and polyglycolic acid (DEXON—available from US Surgical, Inc.). Collagen materials such as COOK SURGISIS, available from Cook Biomedical, Inc. may also be used. Non-resorbable materials including, but not limited to, polypropylene, such as BARD Mesh (available from Davol, Inc.), BARD Soft Mesh (available from Davol, Inc.), SURGIPRO (available from US Surgical, Inc.); TRELEX (available from Meadox Medical); PROLENE (available from Ethicon, Inc.); polyester, such as MERSILENE (available from Ethicon, Inc.); microporous ePTFE, such as SOFT TISSUE PATCH (available from W.L. Gore & Associates, Inc.); and other mesh materials (e.g., available from Atrium Medical Corporation), may be suitable for applications involving permanent correction of tissue or muscle defects. It also is contemplated that the mesh fabric may be formed from multifilament yarns and that any suitable method, such as knitting, weaving, braiding, molding and the like, may be employed to form the mesh material.

For some embodiments employing a separate second layer which may be attached to the first or ingrowth layer, the second layer may be formed from a resorbable layer of SEPRAFILM. A representative and non-limiting sampling of other suitable barrier materials includes a sheet of expanded polytetrafluoroethylene (ePTFE), such as GORETEX available from W.L. Gore & Associates, Inc., having a pore size (submicronal) that discourages tissue ingrowth and adhesion, silicone elastomer, such as SILASTIC Rx Medical Grade Sheeting (Platinum Cured) distributed by Dow Corning Corporation, TEFLON mesh, and microporous polypropylene sheeting (CELGARD). Autogenous, heterogenous and xenogeneic tissue also are contemplated including, for example, pericardium and small intestine submucosa. Absorbable materials, such as oxidized, regenerated cellulose (Interceed (TC7)) may be employed for some applications. It is to be appreciated that any suitable adhesion resistant materials may be used as should be apparent to one of skill in the art.

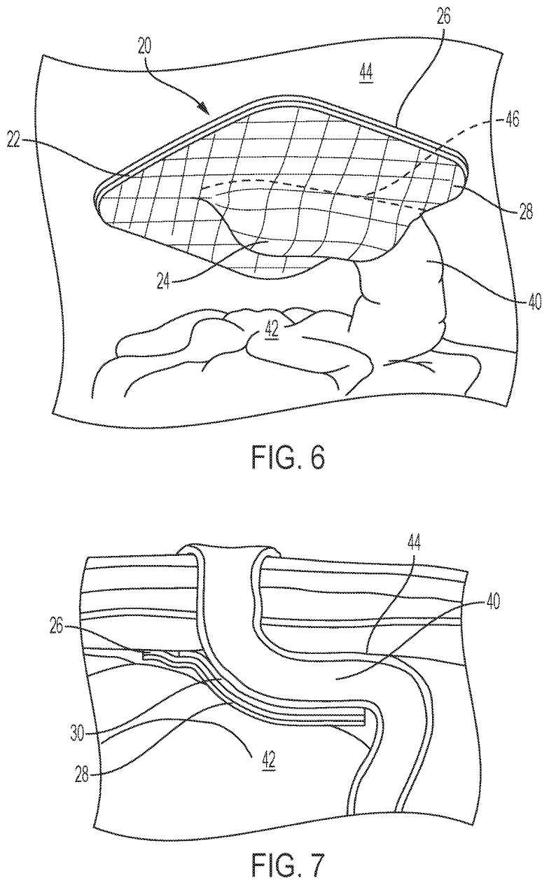

As indicated above, the prosthesis may be suitable for use during a hernia repair procedure, including a parastomal hernia repair. In one embodiment illustrated in FIGS. 6-7, a repair procedure involves the repair of a parastomal hernia using the Sugarbaker technique. As illustrated in FIG. 6, following hernia reduction, sac resection and/or reduction of the stoma trephine to an appropriate size, if necessary, the prosthesis 20 is intraperitoneally placed over the ostomy opening and secured to the fascia. In placing the prosthesis over the ostomy opening, the bowel is lateralized with the preformed channel 24 of the prosthesis being placed over a portion of the lateralized bowel 40. The prosthesis may be fastened to the fascia including the peritoneum and/or tissue overlying the peritoneum. In this manner, the bowel 40 is secured between the prosthesis 20 and the peritoneum, thereby lateralizing the forces which press the bowel ventrally onto the abdominal wall, shifting them from pushing up toward the defect and causing these forces to press ventrally against an intact abdominal wall. If desired, and without limitation, one or more stitches 46 or other suitable fasteners may be placed along the length of the channel to secure the lateralized bowel within the channel. The preformed shape of the channel may help avoid compression of the bowel which may otherwise occur using a flat prosthesis which is placed over the lateralized bowel and fastened along the sides of the bowel.

As shown in FIGS. 6 and 7, the prosthesis 20 may be placed intraperitoneally within the abdominal cavity, via open surgery or a minimally invasive procedure, such as laparoscopic or robotic surgery, and positioned over the ostomy opening with the barrier surface 26 facing the viscera 42 within the abdominal cavity to minimize tissue attachment. The tissue ingrowth surface 24 of the prosthesis faces toward and engages the peritoneum 44 to support functional healing of the hernia via tissue ingrowth. As illustrated in FIG. 7, the lateralized portion of the bowel 40 located within the channel 24 is isolated from the tissue ingrowth surface of the prosthesis by the barrier layer 30 lining the inside surface of the channel facing the bowel to minimize tissue attachment between the prosthesis and the bowel.

As indicated above, the prosthesis may employ a body having any suitable shape for a particular application. In one embodiment illustrated in FIG. 6, the body may have a quadrilateral shape, such as a square or rectangle.

For some applications, it may be desirable to configure the channel so as to reduce pressure against the lateralized bowel and/or to reduce sharp angulation or kinking of the lateralized bowel exiting the channel along the outer periphery of the body. Too much pressure on the bowel exiting the channel could potentially lead to erosion of the body into the intestine. Angulation or kinking of the bowel exiting the channel could potentially lead to intestinal obstruction.

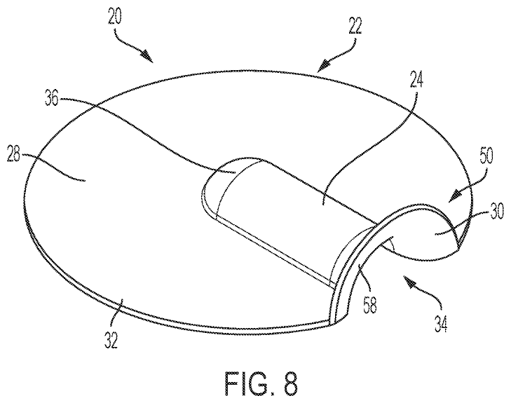

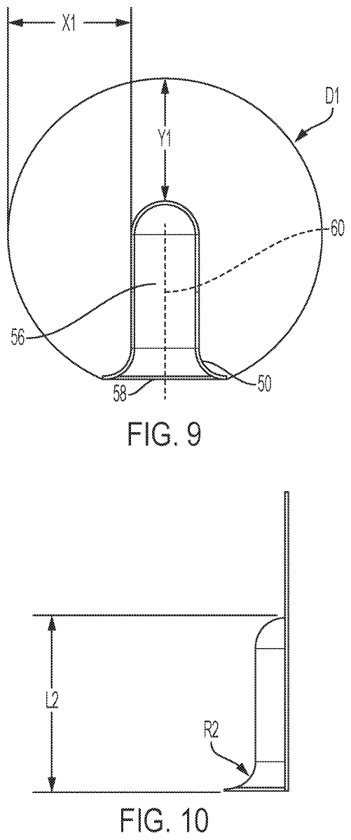

In one illustrative embodiment shown in FIG. 8, the channel 24 may include a flared segment 50 at its open end 34. As illustrated, the flared segment 50 provides a relatively larger opening, as compared to the remainder of the channel. The flared segment gradually decreases in size in a direction toward the closed end of the channel to merge with the primary portion 56 of the channel. In one embodiment, the flared segment 50 may present a relatively large surface area for engaging a bowel exiting the channel. The flared segment may be configured to position its outer periphery 58 at an angle relative to the channel to reduce the likelihood of excessive pressure upon, or excessive angulation of, the lateralized bowel and the outer periphery which could otherwise lead to potential erosion and/or obstruction of the bowel. In one embodiment shown in FIGS. 8-10, the flared segment 50 may be configured to position its outer periphery 58 in a plane perpendicular to the longitudinal axis of the channel.

As indicated above, the body and the channel may be configured to have any shape and/or size suitable for a particular application and/or to accommodate a lateralized bowel of various sizes as should be understood by one of skill in the art. In one embodiment illustrated in FIGS. 8-10, the body 22 may have a circular configuration with a diameter D1 from approximately 5.5 inches (14 cm) to approximately 9.8 inches (25 cm) depending on the size of the bowel being covered by the prosthesis. Similarly, the linear channel 24 may have a length L2 from approximately 2.7 inches (6.9 cm) to approximately 4.9 inches (12.5 cm). The channel may have a radius of curvature R1 (FIG. 4) from approximately 0.6 inches (1.5 cm) to approximately 1.8 inches (4.5 cm) about its longitudinal axis and a full radius of curvature from approximately 0.6 inches (1.5 cm) to approximately 1.8 inches (4.5 cm) about the center of the body to form the closed end of the channel. The flared segment 50 of the channel may have a radius of curvature R2 from approximately 0.6 inches (1.5 cm) to approximately 1.8 inches (4.5 cm). A longitudinal side of the channel may be located a distance X1 away from the outer periphery from approximately 1.6 inches (4 cm) to approximately 3.2 inches (8.1 cm). The closed end 36 of the channel may be located a distance Y1 away from the outer periphery along its longitudinal axis from approximately 1.6 inches (4 cm) to approximately 3.2 inches (8.1 cm).

As indicated above, the prosthesis 20 may be formed with multiple layers of material to provide the desired performance and physical characteristics for covering, reinforcing and/or mending soft tissue and muscle wall defects.

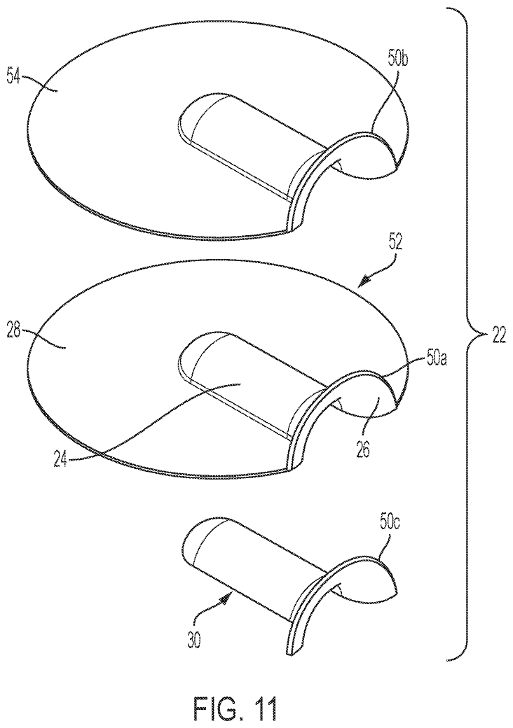

In one illustrative embodiment shown in FIG. 11, the body 22 may include a first or ingrowth layer 52, such as knitted mesh fabric, and a second or barrier layer 54 of adhesion resistant material overlying a surface 28 of the ingrowth layer 52 which is intended to face the viscera, or other organs, tissue and/or muscle, to avoid tissue adhesions to the ingrowth layer. The body may also include a third or channel barrier layer 30 configured to line the inner surface 26 of the preformed channel 24 to isolate the portion of the lateralized bowel from the ingrowth layer 52.

The barrier layers 30, 54 may be separate from and preformed to correspond with the shape of the channel 24 preformed in the ingrowth layer. As illustrated, each layer 30, 52, 54 may include corresponding flared segments 50a, 50b, 50c to provide a relatively larger entrance to the channel, as described above.

The barrier layers 30, 54 may be attached to the ingrowth layer 52 using any suitable fastening technique as should be apparent to one of skill in the art. For example, and without limitation, the ingrowth layer and the barrier layers may be joined together by any one or more of suturing, stitching, in situ polymerization, solvent welding, ultrasonic welding, lamination and/or overmolding.

It is also contemplated that the prosthesis may be placed in other planes within the body. For example, and without limitation, the prosthesis may be utilized for preperitoneal, retrorectus, and/or retromuscular placement. For such applications, it may be desirable for both the first and second surfaces 24, 26 of the body to be tissue infiltratable with an adhesion resistant barrier layer 30 provided only on the inner surface of the channel. In this manner, tissue ingrowth may also occur on the visceral face of the prosthesis.

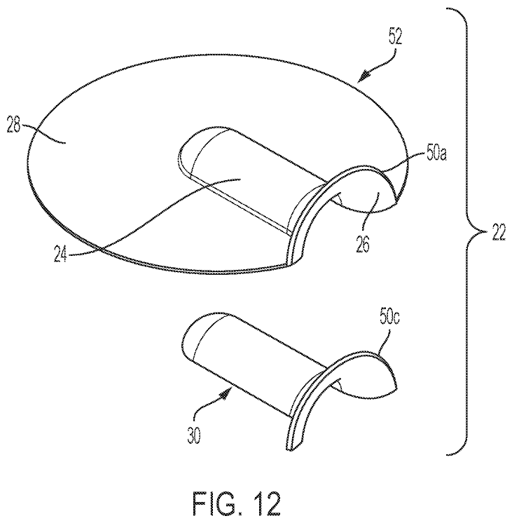

In one embodiment illustrated in FIG. 12, the prosthesis 20 may include a body 22 of implantable, biologically compatible material and a preformed channel 24. The body 22 may include a first or ingrowth layer 52, such as knitted mesh fabric, which may be relatively flexible, thin and light weight and meets the performance and physical characteristics for covering, reinforcing and/or mending soft tissue and muscle wall defects. The body may include a first surface 26 and a second surface 28 opposite the first surface which are both tissue infiltratable. In this manner, and without limitation, the prosthesis may be positioned at a repair site with the first and second surfaces 26, 28 facing soft tissue and/or muscle for receiving tissue ingrowth.

It may be desirable to reduce the incidence of adhesions between the prosthesis and bowel received in the channel. In one embodiment, the prosthesis may include an adhesion resistant barrier 30 to cover the inner surface of the channel and isolate the portion of the lateralized bowel from the first surface 26. In this manner, the barrier layer 30 may reduce, if not eliminate, potential adhesions between the body and the lateralized bowel.

The barrier layer 30 may be separate from and preformed to correspond with the shape of the channel 24 preformed in the ingrowth layer 52. As illustrated, each layer 30, 52 may include corresponding flared segments 50a, 50c to provide a relatively larger entrance to the channel, as described above. The barrier layer 30 may be attached to the ingrowth layer 52 using any suitable fastening technique as should be apparent to one of skill in the art, as described above.

For some applications, it may be desirable to implant the prosthesis using an open surgical procedure during which a surgeon may create an abdominal incision. Thus, in addition to covering the stoma location to repair and/or reduce the incidence of a parastomal hernia, it may be desirable to support the incision. For example, and without limitation, the prosthesis may be used in conjunction with a ventral hernia repair which is accessed via a midline laparotomy incision through a patient's linea alba, which is often the site for a ventral hernia which may develop alone or together with a parastomal hernia.

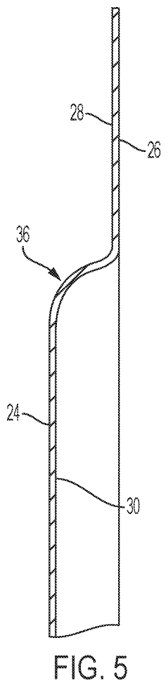

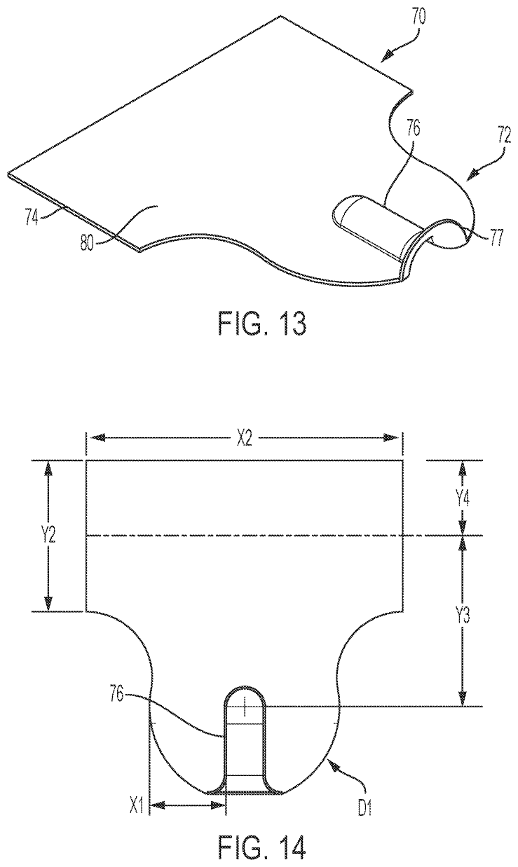

In one embodiment illustrated in FIGS. 13-15, the body 70 of the prosthesis may include a lateral portion 72 and a medial portion 74 extending from the lateral portion. The lateral portion 72 may be configured to cover the site of a stoma and include a preformed channel 76 for receiving a lateralized bowel. In this manner, the lateral portion corresponds with the body 22 of the prosthesis described above and illustrated in FIGS. 1-12. The medial portion 74 may be configured to support a midline laparotomy incision, such as an incision through the linea alba, created during an open surgical procedure.

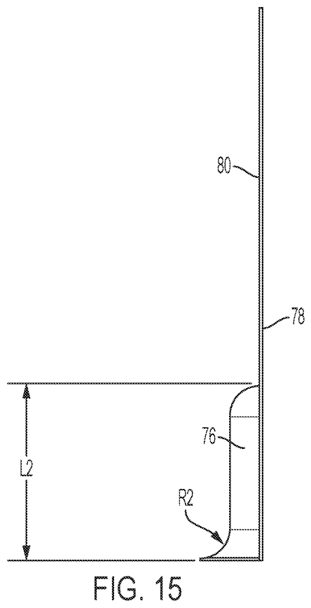

The body may include a first surface 78 (FIG. 15) and a second surface 80 (FIG. 13) opposite the first surface. In one embodiment, the first surface 78 may be tissue infiltratable and the second surface 80 may be adhesion resistant. In this manner, and without limitation, the prosthesis may be positioned at a repair site with the first surface 78 facing soft tissue and/or muscle for receiving tissue ingrowth and the second surface 80 facing the viscera, or other organs, tissue and/or muscle, to avoid tissue adhesions to the body.

In one embodiment, the channel 76 may be configured to receive a portion of a lateralized bowel formed in conjunction with a parastomal hernia repair, such as formed with the Sugarbaker technique. The channel 76 may include an inner side formed by a portion of the first surface 78 and an outer side formed by a portion of the second surface 80. The channel may include a flared segment 77, such as described above, to reduce pressure against the lateralized bowel and/or to reduce sharp angulation or kinking of the lateralized bowel exiting the channel along the outer periphery of the body.

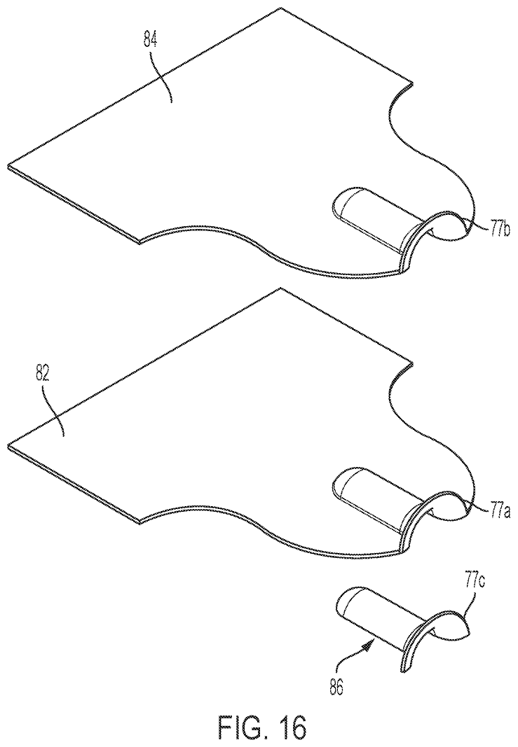

For some applications, it may be desirable to reduce the incidence of adhesions between the prosthesis and bowel received in the channel. In one embodiment, the prosthesis may include an adhesion resistant barrier 84 (FIG. 16) located on the inner side of the channel to isolate the portion of the lateralized bowel from the first surface 26. In this manner, the barrier layer 84 may reduce, if not eliminate, potential adhesions between the body and the lateralized bowel.

In one embodiment, the lateral portion 70 may have a circular shape and the medial portion 72 may have a quadrilateral shape, such as a rectangular shape, which may be aligned with the incision. The medial portion 72 may be configured to have a length X2 in a direction perpendicular to the longitudinal axis of the channel 24 which is greater than the diameter D1 of the lateral portion 70. However, it is to be appreciated that the lateral portion 70 and/or the medial portion 72 may employ any suitable shape and/or size for a particular application as should be understood by one of skill in the art.

In one embodiment illustrated in FIGS. 13-15, the lateral portion 72 may have a generally circular configuration with a diameter D1 from approximately 5.5 inches (14 cm) to approximately 9.8 inches (25 cm) depending on the size of the bowel being covered by the prosthesis. A longitudinal side of the channel may be located a distance X1 away from the outer periphery from approximately 1.6 inches (4 cm) to approximately 3.2 inches (8.1 cm). The closed end 36 of the channel may be located a distance Y1 away from the outer periphery along its longitudinal axis from approximately 1.6 inches (4 cm) to approximately 3.2 inches (8.1 cm).

The channel 76 may be configured have a length L2 from approximately 2.7 inches (6.9 cm) to approximately 4.9 inches (12.5 cm). The channel may have a radius of curvature R1 (FIG. 4) from approximately 0.6 inches (1.5 cm) to approximately 1.8 inches (4.5 cm) about its longitudinal axis and a full radius of curvature from approximately 0.6 inches (1.5 cm) to approximately 1.8 inches (4.5 cm) about the center of the body to form the closed end of the channel. The flared segment 50 of the channel may have a radius of curvature R2 from approximately 0.2 inches (0.5 cm) to approximately 1.8 inches (4.5 cm).

The medial portion 74 may have a quadrilateral shape with a length X2 from approximately 5.5 inches (14 cm) to approximately 11.8 inches (30 cm) and a width Y2 from approximately 3.1 inches (7.9 cm) to approximately 11.8 inches (30 cm). The closed end of the channel may be spaced from the center line Y4 of the medial portion by a distance Y3 from approximately 1.6 inches (4 cm) to approximately 6.3 inches (16 cm).

As indicated above, the body 70 and the channel 76 may be configured to have any shape and/or size suitable for a particular application and/or to accommodate a lateralized bowel of various sizes and/or an abdominal incision as should be understood by one of skill in the art.

As indicated above, the prosthesis 20 may be formed with multiple layers of material to provide the desired performance and physical characteristics for covering, reinforcing and/or mending soft tissue and muscle wall defects.

In one illustrative embodiment shown in FIG. 16, the body 70 may include a first or ingrowth layer 82, such as knitted mesh fabric, and a second or barrier layer 84 of adhesion resistant material overlying a surface of the ingrowth layer 82 which is intended to face the viscera, or other organs, tissue and/or muscle, to avoid tissue adhesions to the ingrowth layer. The body may also include a third or channel barrier layer 86 configured to line the inner surface of the preformed channel 76 to isolate the portion of the lateralized bowel from the ingrowth layer 52.

The barrier layers 84, 86 may be separate from and preformed to correspond with the shape of the channel 76 preformed in the ingrowth layer. As illustrated, each layer 82, 84, 86 may include corresponding flared segments 77a, 77b, 77c to provide a relatively larger entrance to the channel, as described above.

The barrier layers 84, 86 may be attached to the ingrowth layer 82 using any suitable fastening technique as should be apparent to one of skill in the art. For example, and without limitation, the ingrowth layer and the barrier layers may be joined together by any one or more of suturing, stitching, in situ polymerization, solvent welding, ultrasonic welding, lamination and/or overmolding.

For purposes of this patent application and any patent issuing thereon, the indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

The use of “including,” “comprising,” “having,” “containing,” “involving,” and/or variations thereof herein, is meant to encompass the items listed thereafter and equivalents thereof as well as additional items.

It should also be understood that, unless clearly indicated to the contrary, in any methods claimed herein that include more than one step or act, the order of the steps or acts of the method is not necessarily limited to the order in which the steps or acts of the method are recited.

The foregoing description of various embodiments are intended merely to be illustrative thereof and that other embodiments, modifications, and equivalents are within the scope of the invention recited in the claims appended hereto.

1. An implantable prosthesis comprising:
a body of implantable, biologically compatible material, the body having a preformed three-dimensional configuration, the body including a first surface and a second surface opposite the first surface, the first surface being tissue infiltratable, the second surface being adhesion resistant;
a channel preformed on the body, the channel having a three-dimension configuration to receive a portion of a lateralized bowel, the channel including an inner side formed by a portion of the first surface and an outer side formed by a portion of the second surface; and
an adhesion resistant barrier located on the inner side of the channel to isolate the portion of the lateralized bowel from the first surface.

2. The implantable prosthesis according to claim 1, wherein the body includes an outer periphery, the channel extending from the outer periphery to an inner region of the body.

3. The implantable prosthesis according to claim 2, wherein the channel includes an open end at the outer periphery and a closed end opposite the open end.

4. The implantable prosthesis according to claim 1, wherein the body has a maximum width from approximately 5.9 inches to approximately 9.8 inches.

5. The implantable prosthesis according to claim 4, wherein the body has a circular shape with an outer diameter, the maximum width corresponding to the outer diameter.

6. The implantable prosthesis according to claim 1, wherein the channel has a length from approximately 2.9 inches to approximately 4.9 inches.

7. The implantable prosthesis according to claim 1, wherein the channel extends along a longitudinal axis, the channel having a radius from approximately 0.6 inches to approximately 1.8 inches about the longitudinal axis.

8. The implantable prosthesis according to claim 3, wherein the body has a circular shape with a diameter of approximately 5.9 inches to approximately 7.9 inches, the channel having a length along a longitudinal axis of approximately 3.0 inches to approximately 4.0 inches from the outer periphery to a center of the body, the channel having a radius of approximately 0.6 inches about the longitudinal axis.

9. The implantable prosthesis according to claim 8, wherein the body has a diameter of approximately 6.0 inches and the channel has a length of approximately 3.0 inches.

10. The implantable prosthesis according to claim 3, wherein the body has a circular shape with a diameter of approximately 7.1 inches to approximately 8.7 inches, the channel having a length along a longitudinal axis of approximately 3.5 inches to approximately 4.0 inches along the longitudinal axis from the outer periphery to a center of the body, the channel having a radius of approximately 1.2 inches about the longitudinal axis.

11. The implantable prosthesis according to claim 3, wherein the body has a circular shape with a diameter of approximately 8.2 inches to approximately 9.8 inches, the channel having a length along a longitudinal axis of approximately 4.1 inches to approximately 4.9 inches along the longitudinal axis from the outer periphery to a center of the body, the channel having a radius of approximately 1.8 inches about the longitudinal axis.

12. The implantable prosthesis according to claim 3, wherein the closed end has a semi-hemispherical shape with a radius from approximately 0.6 inches to approximately 1.8 inches about the longitudinal axis.

13. The implantable prosthesis according to claim 1, wherein the channel has a perimeter, a portion of the body extending outwardly from the perimeter of the channel having a planar configuration.

14. The implantable prosthesis according to claim 1, wherein the body is resorbable.

15. The implantable prosthesis according to claim 1, wherein the adhesion resistant barrier is resorbable.

16. A method of reinforcing a stoma, repairing a parastomal hernia or preventing a parastomal hernia, the method comprising acts of:
(a) placing a prosthesis intraperitoneally over an ostomy, the prosthesis including a body of implantable, biologically compatible material and a channel preformed on the body, the channel having a three-dimension configuration to receive a portion of a lateralized bowel, the body including a first surface and a second surface opposite the first surface, the first surface being tissue infiltratable, the second surface being adhesion resistant, the channel including an inner side formed by a portion of the first surface and an outer side formed by a portion of the second surface, and an adhesion resistant barrier located on the inner side of the channel
(b) placing the preformed channel over a portion of a lateralized bowel extending from the ostomy with the adhesion resistant barrier isolating the portion of the lateralized bowel from the inner side of the channel; and
(c) securing the prosthesis to fascia.

17. The method according to claim 16, further comprising an act (d) of securing a portion of the prosthesis extending along the channel to the fascia.

18. The method according to claim 16, wherein act (a) includes positioning the first surface facing the fascia and the second surface facing viscera within an abdominal cavity.

19. The method according to claim 16, wherein the body has an outer periphery, the channel including an open end at the outer periphery and a closed end located opposite the open end, and wherein act (a) includes positioning the closed end of the channel over an ostomy trephine.

20. The method according to claim 16, wherein the body has a circular shape with a maximum diameter of approximately 5.9 inches to approximately 9.8 inches, the channel has a length along a longitudinal axis of approximately 2.9 inches to approximately 4.9 inches and a radius of curvature of approximately 0.6 inches to approximately 1.8 inches about the longitudinal axis, the closed end of the channel having a full radius of curvature of approximately 0.6 inches to approximately 1.8 inches.

21. The method according to claim 16, wherein the body is resorbable.

22. The method according to claim 16, wherein the adhesion resistant barrier is resorbable.

23. An implantable prosthesis comprising:
a body of implantable, biologically compatible material, the body having a circular configuration, the body including a first surface and a second surface opposite the first surface, the first surface being tissue infiltratable, the second surface being adhesion resistant;
a channel on the body configured to receive a portion of a lateralized bowel, the channel including an inner side formed by a portion of the first surface and an outer side formed by a portion of the second surface; and
an adhesion resistant barrier located on the inner side of the channel to isolate the portion of the lateralized bowel from the first surface.

24. The implantable prosthesis according to claim 23, wherein the body includes an outer periphery, the channel extending from the outer periphery to an inner region of the body.

25. The implantable prosthesis according to claim 24, wherein the channel includes an open end at the outer periphery and a closed end opposite the open end.

26. The implantable prosthesis according to claim 23, wherein the body has a diameter from approximately 5.9 inches to approximately 9.8 inches.

27. The implantable prosthesis according to claim 23, wherein the channel has a length from approximately 2.9 inches to approximately 4.9 inches.

28. The implantable prosthesis according to claim 23, wherein the channel extends along a longitudinal axis, the channel having a radius from approximately 0.6 inches to approximately 1.8 inches about the longitudinal axis.

29. The implantable prosthesis according to claim 25, wherein the body has a diameter of approximately 5.9 inches to approximately 7.9 inches, the channel having a length along a longitudinal axis of approximately 3.0 inches to approximately 4.0 inches from the outer periphery to a center of the body, the channel having a radius of approximately 0.6 inches about the longitudinal axis.

30. The implantable prosthesis according to claim 29, wherein the body has a diameter of approximately 6.0 inches and the channel has a length of approximately 3.0 inches.

31. The implantable prosthesis according to claim 25, wherein the body has a diameter of approximately 7.1 inches to approximately 8.7 inches, the channel having a length along a longitudinal axis of approximately 3.5 inches to approximately 4.0 inches along the longitudinal axis from the outer periphery to a center of the body, the channel having a radius of approximately 1.2 inches about the longitudinal axis.

32. The implantable prosthesis according to claim 25, wherein the body has a diameter of approximately 8.2 inches to approximately 9.8 inches, the channel having a length along a longitudinal axis of approximately 4.1 inches to approximately 4.9 inches along the longitudinal axis from the outer periphery to a center of the body, the channel having a radius of approximately 1.8 inches about the longitudinal axis.

33. The implantable prosthesis according to claim 25, wherein the closed end has a semi-hemispherical shape with a radius from approximately 0.6 inches to approximately 1.8 inches about the longitudinal axis.

34. The implantable prosthesis according to claim 23, wherein the body is resorbable.

35. The implantable prosthesis according to claim 23, wherein the adhesion resistant barrier is resorbable.

36. A method of reinforcing a stoma, repairing a parastomal hernia or preventing a parastomal hernia, the method comprising acts of:
(a) placing a prosthesis intraperitoneally over an ostomy, the prosthesis including a body of implantable, biologically compatible material and a channel on the body, the channel configured to receive a portion of a lateralized bowel, the body having a circular configuration and including a first surface and a second surface opposite the first surface, the first surface being tissue infiltratable, the second surface being adhesion resistant, the channel including an inner side formed by a portion of the first surface and an outer side formed by a portion of the second surface, and an adhesion resistant barrier located on the inner side of the channel;
(b) placing the channel over a portion of a lateralized bowel extending from the ostomy with the adhesion resistant barrier isolating the portion of the lateralized bowel from the inner side of the channel; and
(c) securing the prosthesis to fascia.

37. The method according to claim 36, wherein act (a) includes positioning the first surface facing the fascia and the second surface facing viscera within an abdominal cavity.

38. The method according to claim 36, wherein the body has an outer periphery, the channel including an open end at the outer periphery and a closed end located opposite the open end, and wherein act (a) includes positioning the closed end of the channel over an ostomy trephine.

39. The method according to claim 36, wherein the body has a maximum diameter of approximately 5.9 inches to approximately 9.8 inches, the channel has a length along a longitudinal axis of approximately 2.9 inches to approximately 4.9 inches and a radius of curvature of approximately 0.6 inches to approximately 1.8 inches about the longitudinal axis, the closed end of the channel having a full radius of curvature of approximately 0.6 inches to approximately 1.8 inches.

40. The method according to claim 36, wherein the body is resorbable.

41. The method according to claim 36, wherein the adhesion resistant barrier is resorbable.

42. An implantable prosthesis comprising:
a body of implantable, biologically compatible material, the body having a preformed three-dimensional configuration, the body including a first surface and a second surface opposite the first surface, the body including an outer periphery; and
a channel preformed on the body, the channel having a three-dimension configuration to receive a portion of a lateralized bowel, the channel including an inner side formed by a portion of the first surface and an outer side formed by a portion of the second surface, the channel extending from the outer periphery to an inner region of the body, the channel including an open end at the outer periphery and a closed end at the inner region, a portion of the channel including a flared segment extending from the outer periphery toward the closed end.

43. The implantable prosthesis according to claim 42, wherein the flared segment of the channel is configured to reduce pressure on the portion of the lateralized bowel at the open end of the channel.

44. The implantable prosthesis according to claim 42, wherein the flared segment decreases in size in a direction from the open end toward the closed end.

45. The implantable prosthesis according to claim 42, further comprising an adhesion resistant barrier located on the inner side of the channel to isolate the portion of the lateralized bowel from the first surface.

46. The implantable prosthesis according to claim 42, wherein the first surface is tissue infiltratable and the second surface is adhesion resistant.

47. The implantable prosthesis according to claim 42, wherein the body includes a first layer of tissue infiltratable fabric and a second layer of adhesion resistant material, the first layer forming the first surface of the body and the second layer forming the second surface of the body.

48. The implantable prosthesis according to claim 42, wherein the channel has a length from approximately 2.9 inches to approximately 4.9 inches.

49. The implantable prosthesis according to claim 42, wherein the channel extends along a longitudinal axis, the channel having a radius from approximately 0.6 inches to approximately 1.8 inches about the longitudinal axis.

50. The implantable prosthesis according to claim 42, wherein the closed end has a semi-hemispherical shape with a radius from approximately 0.6 inches to approximately 1.8 inches about the longitudinal axis.

51. The implantable prosthesis according to claim 42, wherein the flared segment has a radius of curvature from approximately 0.6 inches to approximately 1.8 inches.

52. The implantable prosthesis according to claim 42, wherein the channel has a perimeter, a portion of the body extending outwardly from the perimeter of the channel having a planar configuration.

53. An implantable prosthesis comprising:
a body of implantable, biologically compatible material, the body including a first surface and a second surface opposite the first surface, the body including a lateral portion and a medial portion extending from the lateral portion, the body including an outer periphery; and
a channel preformed on the lateral portion of the body, the channel having a three-dimension configuration to receive a portion of a lateralized bowel, the channel including an inner side formed by a portion of the first surface and an outer side formed by a portion of the second surface, the channel extending from the outer periphery to an inner region of the body, the channel including an open end at the outer periphery and a closed end at the inner region,
the lateral portion configured to be placed at a stoma site to prevent and/or repair a parastomal hernia, the medial portion configured to be placed at a midline abdominal incision when the lateral portion is placed at the stoma site.

54. The implantable prosthesis according to claim 53, wherein the lateral portion has a first configuration and the medial portion has a second configuration which is different from the first configuration.

55. The implantable prosthesis according to claim 54, wherein at least a portion of the outer periphery along the lateral portion has a circular shape.

56. The implantable prosthesis according to claim 54, wherein the medial portion has a quadrilateral shape.

57. The implantable prosthesis according to claim 54, wherein the medial portion has a length from approximately 5.5 inches to approximately 11.8 inches and a width from approximately 3.1 inches to approximately 11.8 inches.

58. The implantable prosthesis according to claim 57, wherein the medial portion has a center line located at 50% of the width, the closed end of the channel is spaced from the center line of the medial portion by a distance from approximately 1.6 inches to approximately 6.3 inches.

59. The implantable prosthesis according to claim 53, wherein a portion of the channel includes a flared segment extending from the outer periphery toward the closed end.

60. The implantable prosthesis according to claim 59, wherein the flared segment of the channel is configured to reduce pressure on the portion of the lateralized bowel at the open end of the channel.

61. The implantable prosthesis according to claim 59, wherein the flared segment decreases in size in a direction from the open end toward the closed end.

62. The implantable prosthesis according to claim 59, wherein the flared segment has a radius of curvature from approximately 0.6 inches to approximately 1.8 inches.

63. The implantable prosthesis according to claim 53, further comprising an adhesion resistant barrier located on the inner side of the channel to isolate the portion of the lateralized bowel from the first surface.

64. The implantable prosthesis according to claim 53, wherein the first surface is tissue infiltratable and the second surface is adhesion resistant.

65. The implantable prosthesis according to claim 53, wherein the body includes a first layer of tissue infiltratable fabric and a second layer of adhesion resistant material, the first layer forming the first surface of the body and the second layer forming the second surface of the body.

66. The implantable prosthesis according to claim 53, wherein the channel has a length from approximately 2.9 inches to approximately 4.9 inches.

67. The implantable prosthesis according to claim 53, wherein the channel extends along a longitudinal axis, the channel having a radius from approximately 0.6 inches to approximately 1.8 inches about the longitudinal axis.

68. The implantable prosthesis according to claim 53, wherein the closed end has a semi-hemispherical shape with a radius from approximately 0.6 inches to approximately 1.8 inches about the longitudinal axis.

69. The implantable prosthesis according to claim 53, wherein the channel has a perimeter, a portion of the body extending outwardly from the perimeter of the channel having a planar configuration.
